(19) United States
(12) Patent Application Publication
(10) Pub. No.: US 2012/0215137 A1
(43) Pub. Date: Aug. 23, 2012

(54) Apparatus and method for improving blood flow in arterioles and capillaries

(76) Inventors: Claudio I. Zanelli, Menlo Park, CA (US); Adrian M. Gianella, Buenos Aires (AR)

(21) Appl. No.: 13/385,236

(22) Filed: Feb. 8, 2012

Related U.S. Application Data
(60) Provisional application No. 61/463,615, filed on Feb. 18, 2011.

(51) Int. Cl. A61N 7/00

(52) U.S. Cl. 601/2

(57) ABSTRACT

Disclosed herein is an apparatus and method which are used to improve blood flow in small vessels such as arterioles and capillaries. Method embodiments of the present invention make use of very high frequency (typically 1 MHZ to 100 MHZ) electrical signals in combination with ultrasonic signals typically falling within the same frequency range, where the frequency of the electrical signals and the ultrasonic signals may be the same or different. The electrical signals and ultrasonic signals are applied to a patient simultaneously, typically at a single contact location which is proximate on the exterior of the body to the arterioles and capillaries where blood flow is impaired.

### FIELD

Embodiments of the invention relate to improvement of blood flow in arterioles and capillaries.

### BACKGROUND

This section describes background subject matter related to the disclosed embodiments of the present invention. There is no intention, either express or implied, that the background art discussed in this section legally constitutes prior art.

Arterioles, which are small diameter blood vessels that extend and branch out from an artery, leading to capillaries. The combination of arterioles and capillaries form what is often referred to as the microcirculation blood vessels. When circulation problems arise in the microcirculation blood vessels, due to the reduced size of their lumen, the general solution is to add chemicals (such as vasodilators, plaque removal compositions, and thrombolytics) to the blood, which may be helpful in relieving the circulation problems.

An article in Endovascular Today, Volume 5, No. 7, July 2006, at Pages 36 -47 describes “Critical Limb Ischemia” (CLI), where most patients have multilevel, multi vessel disease, often with three-vessel tibioperoneal occlusive disease. Limb loss and cardiovascular mortality and morbidity ensue rapidly if CLI is not treated. Treatment options are said to include surgical revascularization, amputation and endovascular intervention. The main treatment goals for CLI are said to be limb preservation, quality-of-life improvement, and a reduction in cardiovascular complications often caused by underlying diffuse arteriosclerosis. The article recommends aggressive medical management, including antiplatelet therapy. Angiotensin-converting enzyme inhibitors are recommended for consideration for hypertension treatment and for their potential to reduce cardiovascular events. Aggressive lipid-lowering therapy and optimal blood glucose control are said to be critically important.

In reaching a diagnosis, delineation of the arterial anatomy to define the extent of arterial occlusive disease is said to be quintessential. Angiography is said to be the gold standard; however, non-invasive evaluation of the extent of arterial occlusion using duplex vascular ultrasound is also said to be a useful starting point. Among the therapies, surgical bypass graft for limb salvage is said to be an effective strategy in some instances. The surgical approach is said to be associated with a prolonged recovery, potential loss of a saphenous vein (which might be needed for future CABG), chronic lower-extremity edema, and worsened symptoms when the graft fails. Complications of infra inguinal bypass are said to include death in 1.3% to 6%, myocardial infarction in 1.9% to 3.4%, wound complications in 10% to 30%, and vein infection in 1.4% of patients. Even when a successful bypass is conducted, amputation of the limb treated is still said to be needed in 5% to 10% of patients.

U.S. Pat. No. 6,871,092 to Piccone, issued Mar. 22, 2005 describes an “Apparatus Designed To Modulate The Neurovegetative System And Integrate Its Action With That Of The Central Nervous System; Applications In The Treatment Of The Vascular System And Orthopaedic Disorders” (Title) The invention is said to relate to an apparatus designed to modulate the neurovegetative system and integrate the neurovegetative action with that of the central nervous system. The method of using the device comprises application of a series of electrical pulses through electrodes located on the epidermis of an area to be treated. The stimulus is controlled directly by the patient in order to achieve better integration with the central nervous system. Vascular disorders resulting from obstruction of the arteries of the legs, heart and brain are said to be effectively treated by inducing vasodilation and increasing blood flow and the production of new blood vessels. The method is also said to improve lesions of the spinal column, especially those affecting the back and neck, and other orthopaedic disorders. (Abstract)

The Piccone invention is said to be based on application of a series of electrical pulses to a patient, whereby a biochemical response can be induced which not only eliminates inflammation from the part of the body treated, but also reduces or eliminates pain. The treatment is also said to have a rapid muscle-relaxant effect, to stimulate vasodilation and Vascular Endothelial Growth Factor (VEGF). (Col. 3, lines 3-9) An apparatus according to the invention is said to include a means designed to generate electrical pulse series having a width from 10 to 40 μsec and intensity from 100 to 170 μAmp, wherein each pulse has a peak that has a width from 7 to 12 nanosecond and a voltage up to 220 Volts. (Col. 3, lines 29-34) Electrodes, one active and one passive (or reference) electrode are said to be applied in different positions, depending on the tissue treated, and regulations of the series of electrical pulses are said to be performable by the patient who may select an increased or reduced voltage. (Col. 4, lines 50-56)

As previously discussed, potential chemical treatments which are available for treating ischemia, these provide less than desirable results in many instances, because the chemicals used have side effects which are not desirable. While the treatment of ischemia using an electrical stimulus through the skin of the kind described in the Piccone reference sounds very attractive, the author discloses that in the case of severe stenosis or arterial obstruction, recurrence of the ischemia symptoms after suspension of the treatment is often due to a deficiency in the development of collateral circulation. In such a case, the treatment must be continued or an arterial bypass performed, which may be followed by new treatment to ensure complete healing of the tissues. (Col. 7, lines 8-14).

There remains a need for a non-invasive treatment method which does not rely on the use of chemicals which are not found to be naturally present in the internal environment which is being treated. There is a need for a treatment which provides a longer lasting, more permanent result.

### SUMMARY

Disclosed herein is a method and apparatus for improving blood flow in small vessels such as arterioles and capillaries.

Method embodiments of the present invention make use of very high frequency (typically 1 MHZ to 100 MHZ) electrical signals in combination with ultrasonic signals typically falling within the same frequency range, where the frequency of the electrical signals and the ultrasonic signals may be the same or different. The electrical signals and ultrasonic signals are applied to a patient simultaneously, typically but not necessarily at a single contact location which is proximate on the exterior of the body to the arterioles and capillaries where blood flow is impaired. One theory about the mechanism of the treatment is that application of the electrical signal causes the endothelium in the area of signal application to generate t-PA. t-PA activates plasminogen to produce the proenzyme plasmin within the arterioles and capillaries which are in the area being treated. The addition of the ultrasound is believed to cause the t-PA to disperse into an area within the vessel where fibrin and accumulated platelets are present in the form of a thrombus which limits blood flow in the arterioles and capillaries. In addition the ultrasound signals may assist in the removal of thrombosis fragments which are generated by break up of a thrombus.

A typical apparatus embodiment of the present invention includes a patient-contacting surface through which both kinds of signal are applied. The patient-contacting surface may be similar to that used in ultrasonic diagnostic techniques, where a transducer is present on a wand which is handled by a person performing the imaging. The contact surface allows the transmission of acoustical signals and includes at least one electrically conductive section that allows the electrical signal to be conducted to the tissue. In one embodiment, the electrical contact surface may be on the exterior portion of a disk surface, by way of example and not by way of limitation, and the acoustic transmission surface may be within an interior portion of the disk surface. In an alternative embodiment, the transducer portion of the contact surface may form the exterior portion of a disk surface, and the electrical contact surface may form an interior portion of the disk surface. In one advantageous embodiment, the whole surface of the transducer is conductive to ultrasonic and electrical signals. To have a complete electrical path, in addition to a first electrical contacting surface of the kind described above, it is necessary to have a second patient contacting surface which provides an electrical return loop after these electrical signals have passed through the patient. The second patient contacting surface may be in the form of a chair or a bed upon which the patient resides, which provides a the current return path because it is connected to ground via electric network mains. Because the signals are of such high frequency, even if there is no direct ohmic contact between this bed or chair to ground, there is enough capacitive connection between the supporting structure (a bed or chair, for example) and the building ground, which at some point is connected to a main ground. The capacitance between the patient and the supporting structure typically ranges from 100 pF to 100 nF, depending on the materials and shapes of the materials involved. For the frequencies described earlier, this represents an impedance that ranges between 0.02 Ohms and 16 kOhms. Since the resistance across a the body is typically in the range of tens of kOhms, the impedance between the patient and the mains ground is not a significant impediment to closing the electric circuit.

### BRIEF DESCRIPTION OF THE DRAWINGS

So that the manner in which the exemplary embodiments of the present invention are attained is clear and can be understood in detail, with reference to the particular description provided above, and with reference to the detailed description of exemplary embodiments, applicants have provided illustrating drawings. It is to be appreciated that drawings are provided only when necessary to understand exemplary embodiments of the invention and that certain well known processes and apparatus are not illustrated herein in order not to obscure the inventive nature of the subject matter of the disclosure.

FIG. 1 shows a cross-sectional schematic 100 of a blood vessel wall 101 which includes an outer layer comprising fibroblasts 102, a layer of smooth muscle cells 104, an intermediary layer of elastica interna 106, and an inner layer of endothelial cells 104.

FIG. 2 shows a cross-sectional schematic of the interior processes taking place within the blood flow channel 201 present within the endothelial layer 210.

FIG. 3 shows a schematic view of the electric signal path when an electric field is applied to a limb or organ of a patient.

FIG. 4 shows a schematic view of the acoustic signal path when an ultrasonic acoustic field is applied to a limb or organ of a patient.

FIG. 5 shows a schematic view of the combined electric and acoustic signal paths when the electric field and ultrasonic acoustic field are applied to the same limb or organ.

FIG. 6 shows the same basic design as shown in FIG. 5, but illustrates an embodiment of the invention in which an electrical signal to the ultrasonic transducer of the apparatus is operated at an attenuation “A”, to produce an ultrasonic field, while an electrical signal to the conductive surface of the apparatus is operated at attenuation “B”.

FIG. 7 illustrates a schematic view of a second embodiment in which a combination of electric and acoustic signal paths are used to apply an electric field and ultrasonic acoustic field to the same limb or organ. In this embodiment a single signal is sent to the electrical contact, and that signal is used to produce both the electric field and acoustic field which are applied to the limb or organ.

### DETAILED DESCRIPTION OF THE INVENTION

As a preface to the detailed description, it should be noted that, as used in this specification and the appended claims, the singular forms “a”, “an”, and “the” include plural referents, unless the context clearly dictates otherwise.

When the word “about” is used herein, this is an indication that the precision of the number provided is within ±50%.

Embodiments of the invention relate to improvement of blood flow in arterioles and capillaries. The method and apparatus which illustrate embodiments of the invention particularly relate to the removal of blood clots from tiny vasculature in a manner which does not affect other parts of the body aside from the localized areas which are treated.

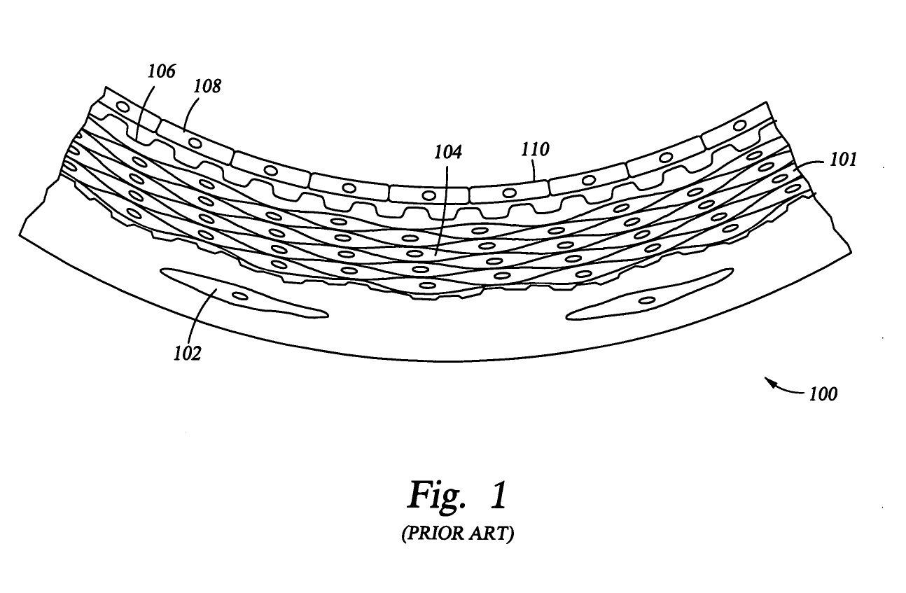

With reference to FIG. 1, which shows a portion of a blood vessel 100, which comprises fibroblasts 102 and other known materials which define the exterior portion of the blood vessel. Overlying the fibroblasts 102 is a layer of smooth muscle cells 104. Overlying the smooth muscle cells is an elastica interna 106 which strengthens the flexibility of the blood vessel, and finally there is an endothelial layer 108 on the internal surface 110 of the blood vessel 100.

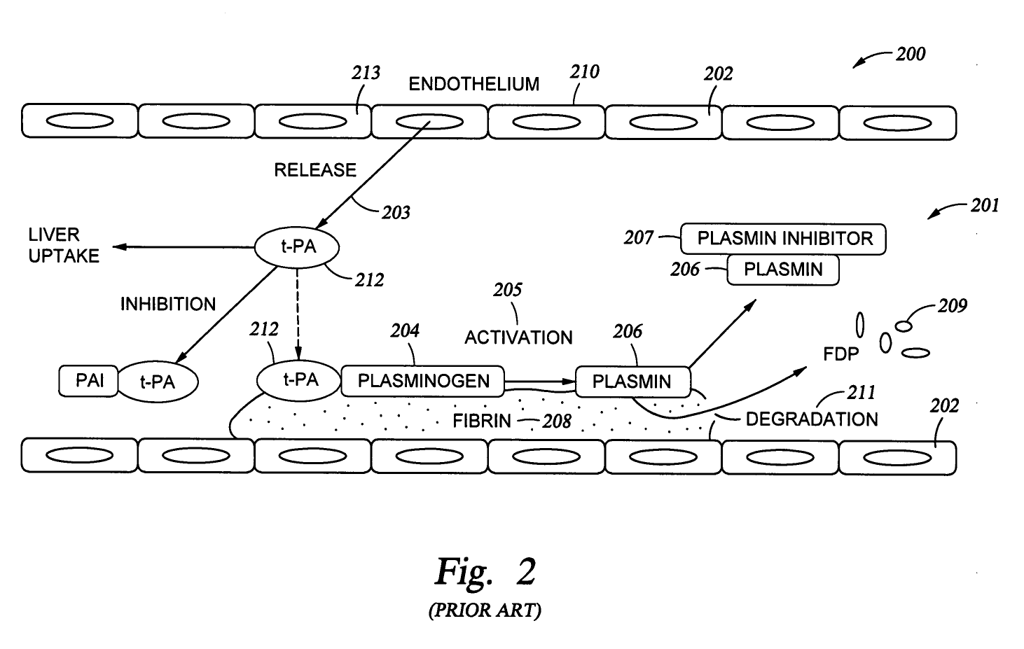

FIG. 2 shows a diagram 200 of activity which takes place inside the internal blood flow channel 201 which is present within the endothelial layer (endothelium) 210. This diagram was published in a Plasminogen Product Monograph by Chromogenix, copyright 1995, which is available on the internet at www.coachrom.com/docs/ . . . /Monographie_Plasminogen.pdf. The description of the diagram 200 recites that: “Plasminogen 204 is the proenzyme of plasmin, whose primary target is the degradation of fibrin 208 in the vasculature. t-PA 212 is the principal activator of plasminogen 204 in blood, while u-PA (not shown) is the predominant activator outside the bloodstream in the extracellular matrix. t-PA 212 is produced by the vascular endothelial cells 213 and is released into the circulation after stimulation. Fibrin 208 regulates its own destruction by providing receptors or binding sites for plasminogen 204 and t-PA 212, thus localizing the action of plasmin . . . ” (identification numbers added in). In the main body of the article entitled “Chromogenic Substrate Assays for Plasminogen and their Diagnostic Relevance”, which accompanies the diagram, at Page 1 in the second paragraph, where the authors teach: “The central component in the fibrinolytic system is the glycoprotein plasminogen, which is produced by the liver and is present in plasma and most extravascular fluids. Plasminogen is a precursor enzyme (zymogen), which, following partial cleavage by a plasminogen activator is converted to its active proteolytic form, plasmin.” All of the methods described to provide for increased production of plasmin are chemical in nature.

There are a number of different theories as to why the apparatus and method of the present invention help improve blood flow in arterioles and capillaries. In addition to the possible stimulation of the production of plasmin, the invention may cause the release of nitrous oxide (NO), which is responsible for vasodilation. Further, once blood flow is improved, this may assist in restoring the health of the vessels themselves, thus maintaining a larger lumen, and in turn restoring the health of the surrounding tissue.

As discussed above, the present inventors developed an apparatus and method which may be used to cause the endothelium to release the t-PA activator which leads to the generation of plasmin, or which may be used to cause the release of nitrogen oxide (NO), for example and not by way of limitation. Once the beneficial compound is produced, it is gently distributed over a localized area which requires treatment.

Method embodiments of the invention make use of very high frequency (typically 1 MHZ to 100 MHZ) electrical signals having amplitudes ranging from about 200 Volts to 700 Volts, an intensity no higher than 700 mW/cm², and a signal duration (pulse duration) ranging from 1μ second to lm second. The electrical signals are applied in combination with ultrasonic signals from a transducer which may have collimated, converging or diverging beams, depending on the size and proximity of the lesion location. The bandwidth of the transducer ranges between 20% and 100%. The acoustic intensity at the focal area may range from about 1 to 700 mW/cm². The time period over which the combined signals is applied may range from about 10 minutes to about 10 hours.

One advantageous embodiment of the apparatus of the invention comprises a contact surface through which both kinds of signal are applied. The electrical signal contact area may be dispersed relative to the ultrasonic signal contact area in a variety of different patterns from a contact surface. Typically the contact surface is relatively flat and metallic, and the transducer is placed on the inside of this surface. It is also possible to use a non-conductive interface for the transducer, by placing the electrical signal contact area around the periphery of the applicator and the transducer contact surface in an the internal portion of contact surface. In an alternative, the transducer contact surface may be around the exterior of a circular disk, with the electrical contact area in the center of the circular disk. Typically the applicator is shaped as to facilitate placement by the operator.

Typically the patient is placed so that there will be a capacitive connection between the patient and a grounding surface, such that the return signal is available to permit continual operation of the electrical and ultrasonic signals. The recommended capacitance over the connection between the patient and the grounding surface may range between 100 pF and 100 nF, for example and not by way of limitation. The impedance is inversely proportional to the frequency of the signal. For example, if the capacitance between the patient and ground is 100pF, at 10 MHZ, with a “patient” resistance of 40 kOhms, all but 0.5% of the signal would be applied to the patient, and the 0.5% would be lost to the capacitive coupling. The surface area of the ultrasound transducer typically ranges from about 2 cm² and about 10 cm². The surface area of the electrical signal transmitter typically ranges from about 0.1 cm² to about 10 cm². As mentioned earlier, the distribution shape of the electrical signal transmitter relative to the ultrasonic transducer may be varied as desired for a particular application.

There are a number of techniques which may be used to determine whether treatment is complete or whether additional treatment is needed. Examples of imaging techniques for blood flow include CT scans, which are a form of X-ray imaging, or DSA (digital subtraction angiography) which also makes use of X-rays. In addition, ultrasound itself may be used, particularly in the form of a Doppler ultrasound scan (Doppler blood flow studies are known as duplex scanning) which is used in diagnosing problems of blood flow, especially arterial disease and venous thrombosis. It is important to mention that restoration of blood flow may also be sensed by looking at the color, and temperature of the treated area, and by the patient assessment of feeling in the limb.

### DESCRIPTION OF THE APPARATUS EMBODIMENTS SHOWN IN THE DRAWINGS

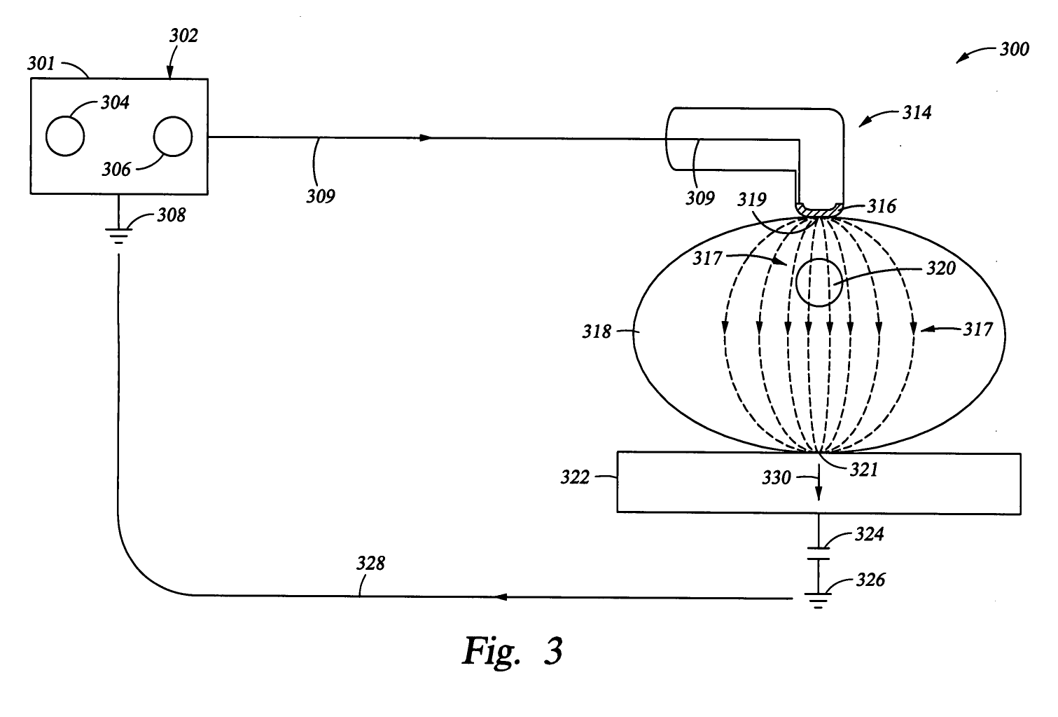

FIG. 3 shows a portion 300 of an embodiment apparatus of the present invention which permits the application of an electrical field 317 to a target 320 to be therapeutically treated. The pulse repetition frequency control 304 and amplitude control 306 for the electrical signal 309 used to generate electrical field 317 are included within a pulse generator 302. The electrical signal 309 travels to an applicator 314, which includes a conductive surface 316, which is used to apply the electrical signal 309 to a first surface 319 of a patient's body 318 (a leg surface, for example) which is adjacent to the therapy target 320 within the patient's body. The applied electrical signal 309 produces an electric current 317 in the area of the therapy target 320. At least the portion of the patient to be treated is in contact at a second surface 321 with a support structure 322 which provides a coupling capacitance 324 with building ground 326. The building ground is in communication 328 with the building mains ground (building main grounding bar) 308. As the pulse generator 302 is also connected to the mains ground 308, the circuit loop is closed.

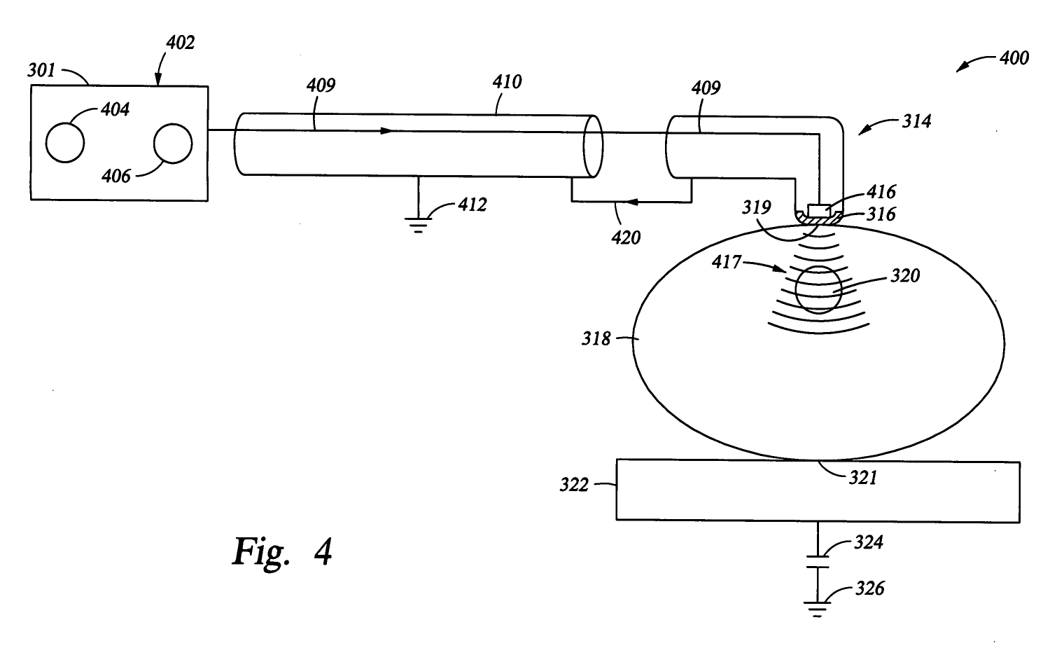

FIG. 4 shows a portion 400 of an embodiment apparatus of the present invention depicting the generation and application of an ultrasonic field 417 to the target 320 to be therapeutically treated. The pulse repetition frequency control 404 and the amplitude control 406 for the electrical signal 409 (used subsequently to create an ultrasonic field 417) are included within a pulse generator 402. The ultrasonic wave 417 is generated by a transducer 416 when the signal 409 is applied to transducer 416. The ultrasonic wave 417 travels through a matching layer, wear plate, or lens 316. In the embodiment shown in FIG. 4, the transducer 416 is present behind a conductive surface 316 of applicator 314. The ultrasonic signal is not significantly affected by the direction in which the current flows. The circuit loop is completed by flow path 420 which leads to ground 412.

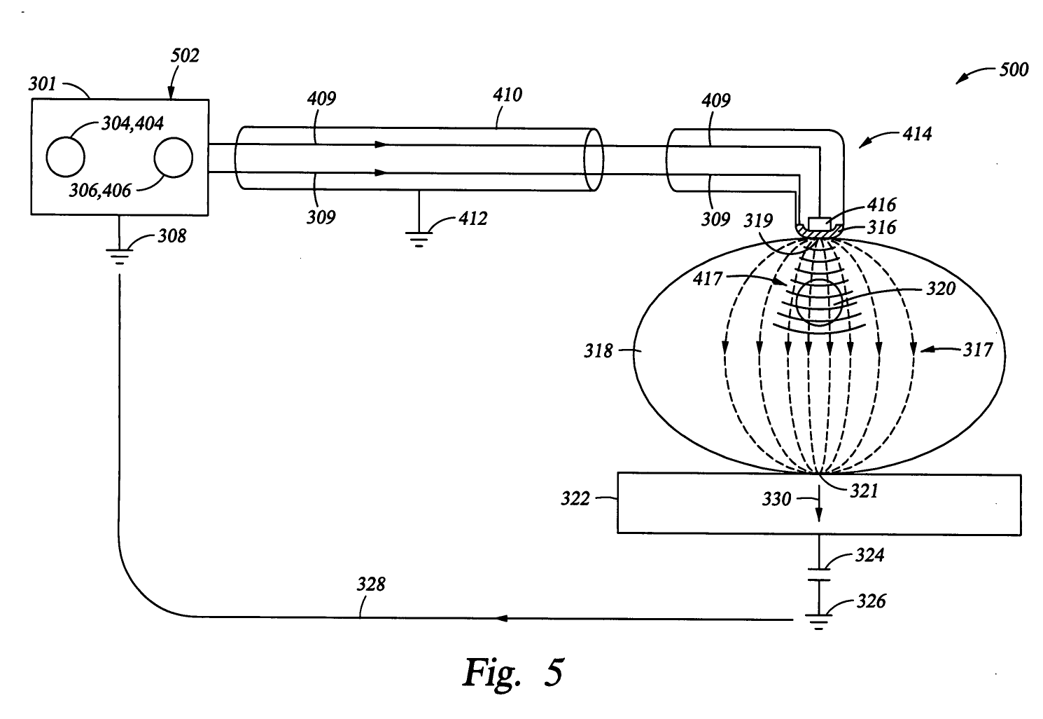

FIG. 5 shows an embodiment apparatus 500 in a manner which illustrates application of the combination of electrical current field 317 and ultrasonic field 417, as required for the therapeutic treatments of the kind described herein. The pulse generator 502 produces an electrical signal 309 which is used to produce electric current field 317 which flows through the target region 320 to the support frame 322, then through capacitance 324 to ground 326, thus completing the current loop. The electrical signal 309 depends on the setting of the pulse repetition frequency control 304 and the setting of the amplitude control 306. Simultaneously, pulse generator 502 produces an electrical signal 409 which is used to produce ultrasonic wave field 417. The electrical signal 409 which is produced depends on the pulse repetition frequency control 404 and the setting of the amplitude control 406. The pulse repetition frequency control 304 and amplitude control 306 for the electrical signal 309 and the pulse repetition frequency control 404 and amplitude control 406 for the ultrasonic electrical signal 409 may also be present in the single housing 301, for functional and operational efficiency. For convenience, and to provide an additional source for grounding 412, electrical signals 309 and 409 may be contained in a shielded conduit 410.

The electrical signal 309 travels to the signal applicator 314, which includes a conductive surface 316, which is used to apply the electrical signal 309 to a first surface 319 of a patient's body 318 (a leg surface, for example), and to produce an electrical field 317 passing through the therapy target 320. The electrical signal 409 travels to the transducer 416, which is in contact with the first surface 319 of a patient's body 318, to produce an ultrasonic field 417 passing through the therapy target 320. At least one portion of the patient to be treated is in contact at a second surface 321 with a support structure 322 which provides a coupling capacitance 324 with building ground, as previously described.

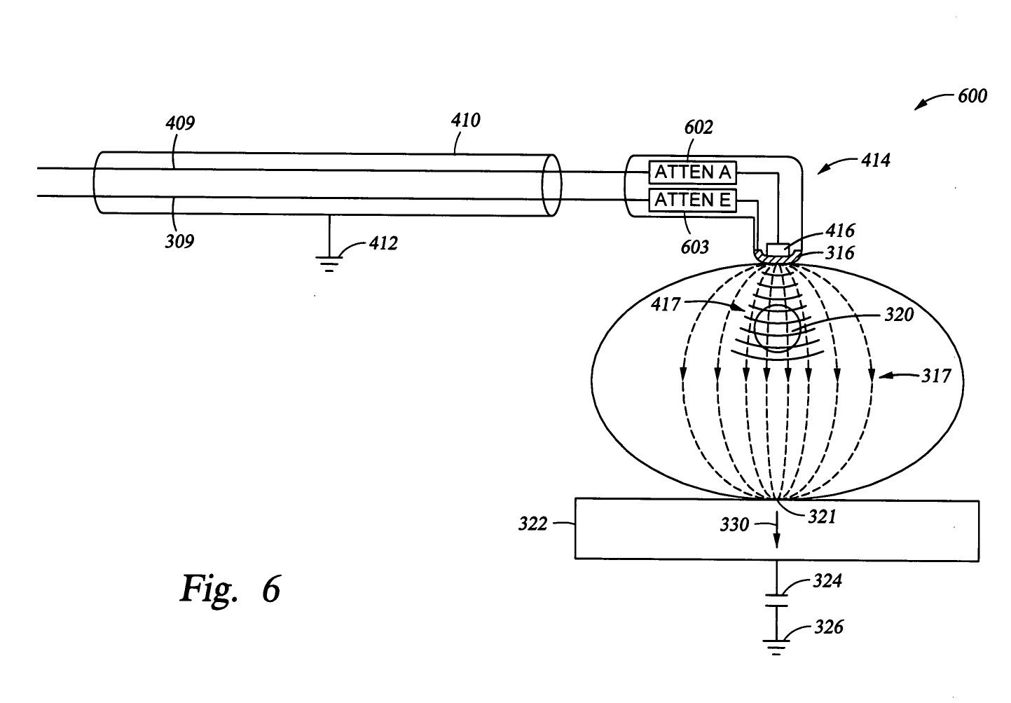

FIG. 6 shows the same basic design as shown in FIG. 5, but illustrates an embodiment of the invention in which an electrical signal 409 to an ultrasonic transducer 416 is operated at an attenuation “A” to produce an ultrasonic field 417, while an electrical signal 309 to a conductive surface 316 is operated at an attenuation “B” to produce an electrical field 317.

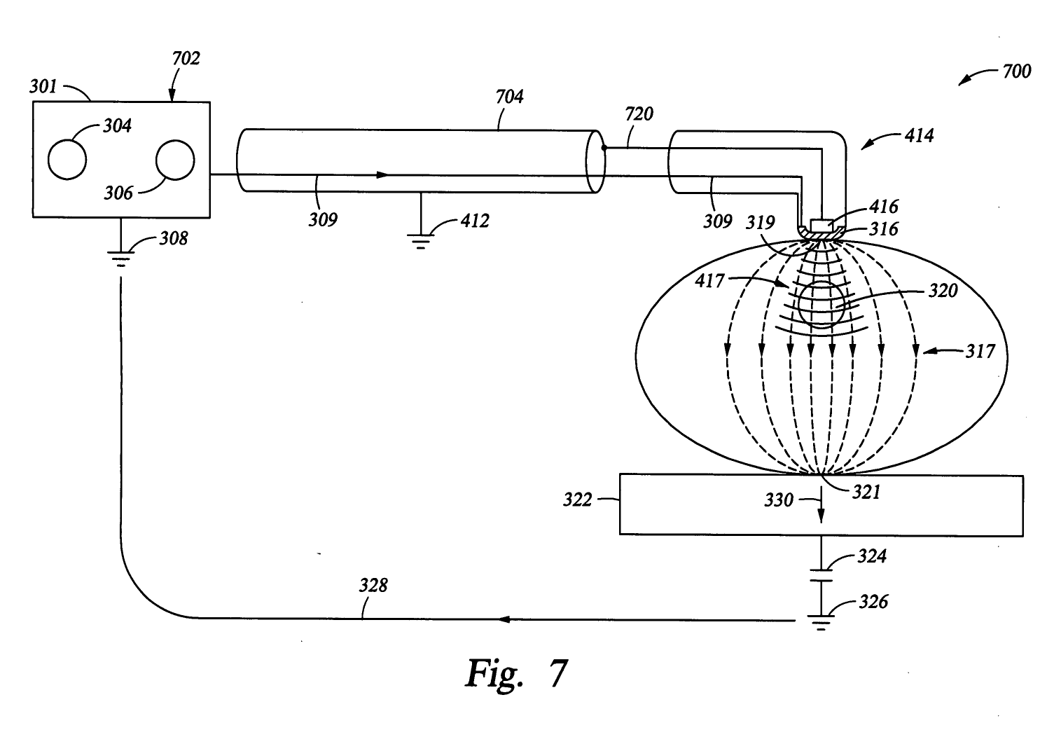

FIG. 7 illustrates another embodiment which requires fewer elements to produce electrical current field 317 and ultrasonic wave field 417, but which provides less variability in terms of pulse repetition frequency and amplitude control. A single pulse repetition frequency control 304 and amplitude control 306, present within pulse generator 702, are used to produce a single electrical signal 309 which is used to produce electrical current field 317 and ultrasonic wave field 417, as the electrical signal 309 generates electrical current field at conductive surface 316, and simultaneously passes through conductive surface 316 to ultrasonic transducer 416 to produce ultrasonic wave field 417. The ultrasonic circuit loop is closed by flow path 720 which leads to ground 412.

While the foregoing is directed to embodiments of the present invention, other and further embodiments of the invention may be devised in view of the present disclosure, without departing from the basic scope of the invention, and the scope thereof is determined by the claims which follow.

I claim:

1. A method of improving blood flow in arterioles and capillaries, comprising:
applying at least one electrical current field to a body surface of a patient to be treated;
simultaneously applying at least one ultrasonic wave field to a body surface of said patient to be treated, said electrical current field and said ultrasonic wave field being applied in a manner such that said electrical current field and said ultrasonic wave field overlap within a location of said patient's body in which said blood flow in arterioles and capillaries is to be improved.

2. A method in accordance with claim 1, wherein said electrical current field and said ultrasonic wave field are applied from a common surface which is placed on a body surface of said patient to be treated.

3. A method in accordance with claim 1 or claim 2, wherein said electrical current field is set by adjusting a pulse repetition frequency control device, a frequency control device, or a combination thereof.

4. A method in accordance with claim 1 or claim 2, wherein said ultrasonic wave field is set by adjusting a pulse repetition frequency control device, a frequency control device, or a combination thereof.

5. A method in accordance with claim 1 or claim 2, wherein said electrical current field and said ultrasonic wave field are set by adjusting a single pulse repetition frequency control device, a single frequency control device, or a combination thereof.

6. An apparatus which is used to improve blood flow in arterioles and capillaries, comprising:
at least one electrical current field generation device;
at least one ultrasonic wave field generation device;
at least one conductive surface which receives a signal from said at least one electrical current field generation device, receives a signal from said at least one ultrasonic wave field generation device, or a combination thereof; and,
at least one structure which is used in combination with said at least one conductive surface to complete an electric circuit with respect to said at least one electrical current field generation device and with respect to said at least one ultrasonic wave field generation device.

7. An apparatus in accordance with claim 6, wherein said at least one electrical current field generation device is in communication with a pulse repetition frequency control device or an amplitude control device, or a combination thereof.

8. An apparatus in accordance with claim 6, wherein said at least one ultrasonic wave field generation device is in communication with a pulse repetition frequency control device or an amplitude control device, or a combination thereof.

9. An apparatus in accordance with claim 6, wherein an electrical current field generation device and an ultrasonic wave field generation device are in communication with the same pulse frequency control device, or amplitude control device, or combination thereof.

10. An apparatus in accordance with claim 6, wherein said at least one conductive surface and said at least one structure used to complete said electric circuits are configured to be placed on opposite sides of a body location which contains said arterioles and capillaries to be treated.
